(19) United States
(12) Patent Application Publication
(10) Pub. No.: US 2002/0146424 A1
(43) Pub. Date: Oct. 10, 2002

(54) Polyphenolics for enhancing endothelial cell-mediated fibrinolysis

(76) Inventors: Raymond L. Benza, Birmingham, AL (US); Francois M. Booyse, Birmingham, AL (US); Michael L. Aikens, Birmingham, AL (US)

Correspondence Address: Glenna Hendricks, Esq., P.O. Box 2509, Fairfax, VA, 22031-2509, US

(21) Appl. No.: 10/078,267

(22) Filed: Feb. 20, 2002

Related U.S. Application Data
(60) Provisional application No. 60/269,351, filed on Feb. 20, 2001.

(51) Int. Cl. A61K 39/00; A61K 39/38

(52) U.S. Cl. 424/184.1

(57) ABSTRACT

This invention to provides means of achieving cardiovascular protective effects by administration of fibrinolytic activity increasing amounts of catechin, epicatechin, quercetin and/or resveratrol or their complexes individually or in combination without administration of ethanol.

This application takes priority for U.S. Provisional Patent Application No. 60/269,351, filed Feb. 20, 2001.

### FIELD OF THE INVENTION

This invention relates to use of polyphenols, including metabolically or synthetically modified forms, to promote systemic fibrinolysis in the prevention/regression and treatment of atherogenesis and its atherothrombotic consequences, including myocardial infarction, unstable angina, claudication, acute limb ischemia and thrombotic cerebrovascular events.

### BACKGROUND OF THE INVENTION

Previous studies suggest that the moderate consumption of red wine is associated with lower coronary heart disease (CHD) related mortality. Even though the mechanism by which the cardioprotection occurs has not been fully elucidated, this reduction in cardiovascular mortality is believed to be, in part, due to components found in red wine. Low ethanol levels have been shown to have various effects on vascular endothelial cell (EC) mediated fibrinolysis. ECs play a key role in maintenance of hemostasis by synthesis/regulation of plasminogen activators (PAs), tissue type-PA (t-PA) and urokinase type-PA (u-PA) and their respective receptors. These fibrinolytic proteins interact to localize and regulate fibrinolysis on the EC surface. Therefore, systemic factors that will affect EC PAs and/or receptors and increase fibrinolysis may reduce the risk for thrombosis, CHD and myocardial infarction (MI).

A number of both in vivo and in vitro studies have demonstrated that alcohol can provide a cardioprotective effect by promoting fibrinolytic activity. For example, a nurse's health study and a physician's health study provided epidemiological data which indicated that moderate alcohol consumption significantly lowered the incidence of heart disease-related events and increased the plasma t-PA levels in subjects when compared to age-matched non-drinkers. These findings suggest that cardioprotective effect was a result of increased fibrinolysis due to elevated levels of plasma t-PA. In addition, recent in vitro studies using cultured human ECs have confirmed an ethanol-induced rapid (direct effect, after a 15 minute exposure) and sustained (24 hours after a 1 hour exposure) increase in fibrinolytic activity. Furthermore, it has been demonstrated that this increased surface-localized fibrinolytic activity is due, in part, to transcriptional increase in t-PA gene expression and urokinase-type plasminogen activator (u-PA) gene expression as well simultaneous down-regulation of PA inhibitor type 1 (PAI-1) gene expression. Another effect of moderate alcohol on the fibrinolytic system includes the transcriptional up-regulation of the candidate plasminogen receptor annexi II and u-PA receptor, u-PAR, gene expression.

The question remains whether other non-alcoholic components of red wine such as the polyphenolic compounds which include resveratrol, quercetin, catechin and epictechin, confer an additional cardioprotective effect above and beyond that expected by the presence of ethanol. A growing body of experimental and clinical evidence suggests red wine polyphenolics may provide an additive cardioprotective effect through decreasing low-density lipoprotein (LDL) oxidation, inhibiting of cyclo-oxygenase and lipoxygenase (hence, reduced thrombotic tendencies), and decreasing platelet aggregation. Polyphenolics increase NO and endothelium-dependent vasorelaxation and reduce atherosclerotic lesion areas in cholesterol-fed mice and rabbits.

There are many persons, such as alcoholics, minors and diabetics, who can not or should not consume wine. Many persons, for religious reasons, do not ingest ethanol-containing beverages. Furthermore, persons such as truck drivers and airline pilots often should refrain, for long periods of time, from drinking alcohol-containing beverages. Studies that have been done to determine possible beneficial effects of de-alcoholized red wine in humans showed enhanced plasma antioxidant capacity.

A number of studies have compared the effects of red wine with those of grape juice on the cardiovascular system. These studies have shown red wine has the more potent effect. For example, in one study consumption of grape juice, unlike red wine, did not alter ADP-induced platelet aggregation or plasma thromboxane levels.

### SUMMARY OF THE INVENTION

It is the purpose of this invention to provide cardiovascular protective effects by administration of fibrinolytic activity increasing amounts of catechin, epicatechin, quercetin and/or resveratrol or their complexes individually or in combination without administration of ethanol.

### DESCRIPTION OF THE INVENTION

It is the purpose of this invention to provide beneficial protection from coronary heart disease and other vascular diseases which result in the pathologic formation of intravascular thrombi and/or from atherosclerosis, by administration of compositions containing protective components found in wine while avoiding the alcoholic content of wine. Studies have demonstrated that the polyphenolic components, quercetin, resveratrol, catechin and epicatechin, active agents found in red wine, can promote endogenous fibrinolytic activity, thus favorably altering the atherosclerotic process and enhancing the dissolution of pathologic thrombi. These agents facilitate this favorable state by transcriptionally up-regulating t-PA and u-PA mRNA gene expression. The net result of these activities is a profibrinolytic state on the surface of the atherosclerotic vessel. Hence, these phenols may be useful therapeutically both as maintenance therapy in patients with atherosclerosis to prevent surface thrombus formation or plaque regression and/or as adjuvant therapy during acute events such as myocardial infarction, unstable angina, nonhemorrhagic stroke or threatened limb ischemia. These agents also relax blood vessels, which also results in benefit to the patient suffering from cardiovascular disease.

The anti-clotting activity associated with the polyphenolics used in the method of the invention is similar to that obtained by administration of aspirin. However, aspirin effects the platelets and causes lowering of hematocrit. The polyphenols, as indicated above, act by facilitating profibrinolytic activity. Furthermore, it is possible to avoid the untoward gastrointestinal effects of aspirin using the methods of the invention.

In red wine, catechin is present in the highest concentration (about 500 μM) and resveratrol in the lowest concentration (4 μM). In vitro studies were conducted to determine whether polyphenolic compounds alone (not in conjunction with alcohol) affect the net expression of surface-localized cultured HUVEC fibrinolytic activity. The desired effect is distinct and separate from ethanol and antioxidant effects resulting from intake of grape-derived beverages. In-vivo studies in animals support the powerful profibrinolytic potential of these polyphenolics. In rats fed these phenols, a favorable antithrombogenic environment was created on the surface of the aortic endothelium. Together, these in vitro and in vivo studies show that the combination or individual use of these phenols can benefit patients with atherosclerosis, particularly those with coronary artery disease, and can be useful for treatment of myocardial infarction or unstable angina.

Materials and Methods:

Purified high molecular weight Mᵣ 54-kDa two-chain u-PA (tcu-PA) was a generous gift from GreenCross. The anti-t-PA and anti-u-PA IgG was from American Diagnostics. The polyphenolics, namely, quercetin (rutinoside), resveratrol, catechin and epicatechin were from Sigma. The Wine Institute, California, provided red wine and de-alcoholized red wine. Transcriptional vector (pGEM-3Z), KpnI, SphI, RNase A and T4 DNA Ligase were purchased from Promega Inc. (Madison, Wis.).

Rats used in in vivo studies were Sprague Dawley rats weighing 250 to 300 grams.

Cell Culture

Human umbilical vein ECs (HUVECs) were obtained from fresh (discarded) umbilical cords by mild collagenase treatment (type 1, CLS) by the method of Jaffe et al (J. Clin Invest 52:2745-2756 (1973)) as modified by Li et al (Arterioscler Thromb Vasc Biol 15:410-419 (1995)). ECs were seeded into human fibronectin-coated Petri dishes (9.6 cm²) or plastic flasks (25 cm²) and grown to confluency. Cultures were maintained at 37° C. in a humidified 95% air, 5% CO₂ atmosphere and re-fed every 48 hours with complete culture medium consisting of Medium 199 (M199) powdered medium containing L-glutamine and Earle's salt, 0.025 M HEPES buffer (pH 7.4), 0.0002 M fresh L-glutamine, 100 units/ml of penicillin, 100 μg/ml streptomycin, 10% heat-deactivated FBS, 90 μg/ml heparin and 50 μg/ml of unpurified ECGF. All polyphenolic induction studies for measurement of fibrinolytic activity assays (plasmin generation) were performed with pooled (4 to 6 umbilical veins), serially subcultured (2nd and 3rd passages), postconfluent (2 to 3 days after reaching stable confluencey density, 8.9 to 9.2×10⁵ cells/cm²), HUVECs grown in 96-well plates. Cells were routinely counted using phase microscopy and a 0.5 mm×0.5 mm counting reticule. Individual HUVEC cultures were routinely examined for their purity and characterized as ECs by their uptake of Dil-Ac-LDL and typical monlayer “cobblestone” tight-packing morphology. Only individual cultures with 95% identifiable ECs were used in these studies.

Iodination of Glu-Pmg

Purified human Glu-Pmg (100 μg) in 0.25 ml of Dulbecco's phosphate buffered saline (DPBS) was iodinated with 250-300 μCi of Na¹²⁵I by the Iodo-Bead method. The reaction was terminated by removal of the Iodo-Beads from the sample and free iodine was removed by gel filtration chromatography using a Sephadex G-25 column. Specific activities of the ¹²⁵I-labeled Glu-Pmg were determined at 1.5 to 2×10⁶ cpm/μg.

Preincubation (Induction) of Cultured HUVECs with Alcohol

All alcohol induction studies were conducted with confluent cultured HUVECs (in 96-well plates) preincubated in the absence/presence of varying concentration of alcohol (0-0.1%, v/v) for one hour at 37° C., washed two times in DBPS, re-fed with complete 10% serum containing medium and then further incubated at 37° C. in the absence of alcohol for 24 hours. surface-localized fibrinolytic activity was measured as the conversion of ¹²⁵-labeled Glu-Pmg levels after 24 hours.

Preincubation (Induction) of Cultured HUVECs with Red Wine, De-Alcoholized Red Wine and Grape Juice

All studies were conducted with confluent cultured HUVECs. The cells were preincubated in the absence/presence of varying dilutions (1:1000, 1:500, 1:250, 1:125, 1:62.5) of red wine, de-alcoholized red wine or grape juice for 1 hour at 37° C., washed two times with DPBS, re-fed with complete 10% serum-containing medium, and then further incubated at 37° C. in the absence of these compounds for 24 hours. Surface-localized fibrinolytic activity was measured as the conversion of ¹²⁵-labeled Glu-Pmg levels after 24 hours.

Preincubation (Induction) of Cultured HUVECs with Red Wine Polyphenolic Compounds (Resveratrol, Quercetin, Catechin and Epicatechin)

All polyphenolic induction studies were conducted with confluent cultured HUVECs (in 96-well plates). The cells were preincubated in the absence/presence of varying concentrations (0.001-10 μM) of catechin, epicatechin, quercetin or resveratrol for 1 hour at 37° C., washed two times with DPBS, re-fed with complete 10% serum-containing medium, and then further incubated at 37° C. in the absence of these compounds for 24 hours. Surface-localized fibrinolytic activity was measured by the direct conversion of cell-bound ¹²⁵I-labeled Glu-Pmg hours after the initial induction period.

Analysis of Surface-Localized Fibrinolytic Activity in Cultured HUVECs

Surface-localized fibrinolytic activity was measured by the direct conversion of single-chain ¹²⁵I-labeled Glu-plasminogen (Pmg) by endogenous receptor-bound PAs to 2 chain ¹²⁵I-labeled plasmin. The generation of either ¹²⁵I-labeled plasmin Mᵣ 20-kDa light or Mᵣ 83-kDa heavy chain formation was quantitated by phosphorimaging autoradiography after SDS-PAGE under reduced conditions. Briefly, HUVECs pretreated in the absence/presence of ethanol, read wine, dealcoholized red wine, grape juice or wine polyphenolics were equilibrated with DPBS at 4° C. for 15 minutes followed by the subsequent addition of saturating levels of ¹²⁵I-labeled GluPmg (2 μM) containing 1,000 KIU/ml of aprotinin and 1% BSA at 4° C. for 20 minutes. The cultures were then placed in a water bath at 37° C. to initiate the receptor-bound endogenous PA-mediated conversion of ¹²⁵I labeled Glu-Pmg to ¹²⁵I-labeled plasmin. Reactions were stopped after 1 and 10 minutes by the rapid addition of 40 μl of hot (56° C.) solubilizing buffer (4% sodium dodecyl sulfate (SDS), 10% glycerol and 0.2M Tris-HCl, pH 6.8). The total solubilized contents of each well containing generated ¹²⁵I-labeled plasmin were removed and reduced by the addition of 5% B-mercaptoethanol and boiling for 5 minutes. Reduced samples containing the ¹²⁵I-labeled Glu-Pmg and ¹²⁵I-labeled plasmin were analyzed by SDS-PAGE using a 1.8×82×74 mm polyacrylamide 4-15% gradient gel. The radioactivity content of either the ¹²⁵I-labeled plasmin light- or heavy-chains was determined by quantitative phosphorimaging autoradiography.

SDS-PAGE and Quantitative Phosphorimaging Autoradiographv

After electrophoresis, gels were dried and exposed in phosphorimaging cassettes for 16-18 hours. The amount of remaining ¹²⁵I-labeled Glu-Pmg and newly generated ¹²⁵I-labeled Mᵣ 20-kDa plasmin light chain in each individual gel was quantitated by measuring the radioactivity content in each band by phosphorimaging autoradiography, using a Molecular Dynamics Series 425F Phosphorimager in combination with ImageQuant software. The radioactivity content in each band was then converted to a plasmin concentration by comparing the radioactivity content of each individual band with the radioactivity content of plasmin ¹²⁵I-labeled M 20-kDa light chain (standard) derived from a known amount of fully converted ¹²⁵I-labeled Glu0Pmg (1.0 μg) in DPBS containing 1000 KIU/ml of aprotinin by incubation with two-chain urokinase-type plasminogen activator (tcu-PA) for 1 hour at 37° C.

Analysis of Data

All of the data were expressed as the means ±SD of triplicate experiments performed in each assay and analyzed by Student's t test. Data with p<0.05 were taken to represent statistically significant differences in experimental results.

Findings:

Dose Dependent Comparison of Ethanol and Its Dilution Equivalent in Red Wine on Surface-Localized Fibrinolytic Activity in Cultured HUVECs

To compare ethanol with its dilution equivalents in red wine effects on EC surface-localized fibrinolytic activity, cultured HUVECs were preincubated with ethanol at varying concentration (0-0.1%, v/v) of ethanol for 1 hour at 37° C. The cells were then rinsed and further incubated with 10% serum containing complete medium in the absence of ethanol for 24 hours. This was followed by addition of ¹²⁵I-labeled Glu-Pmg, and fibrinolytic activity determination as described above. Ethanol doses equivalent to concentrations present in red wine increased fibrinolytic activity in a dose-dependent manner ranging from about 1.5 fold (p=0.05) at 0.05% to about 3 fold at 0.1% (p=0.0055) compared to culture mediums.

Fibrinolytic Activity in Cultured HUVECs

To determine the sustained effect of red wine, dealcoholized red wine and grape juice on fibrinolytic activity, confluent cultured HUVECs were preincubated (1 hour) at 37° C. in the absence/presence of varying dilutions (1:1000; 1:500; 1:250; 1:125; 1:62.5) of the red wine, de-alcoholized wine or grape juice. The cells were then rinsed and incubated in 10% serum containing complete culture medium in the absence of these compounds for 24 hours before fibrinolytic activity was determined using a direct ¹²⁵I-labeled Glu-Pmg activation assay as described above. Red wine induced a significant dose dependent increase in sustained (24 hours after induction) fibrinolytic activity compared to control cultures (42.0±2.8 pmol/well). This increase ranged from about 1.5 fold (49.0±8.5 pmole/well) (p=0.039) at a 1:500 dilution to about 3.2 fold (128.0±2.98 pmole/well) (p=0.005) at a 1:62.5 dilution. De-alcoholized red wine also showed a dose-dependent increase in fibrinolytic activity compared to control cultures (42.0±2.8 pmol/well) ranging from about 1.5 fold (50.0±4.2 pmole/well (p=0.03) at 1:250 dilution to about 1.0 fold (92.5±7.8 pmole/well at a 1:62.5 dilution (p=0.01). There was no significant change in fibrinolytic activity with grape juice compared to control cultures.

Sustained Effect of Polyphenolic Compounds (Resveratrol. Quercetin, Catechin and Epicatechin) on Surface-Localized Fibrinolytic Activity in Cultured HUVECs

To demonstrate a sustained increase in fibrinolytic activity with red wine derived polyphenolics, quercetin, resveratrol, catechin, and epicatechin, ECs were preincubated in the presence/absence of each polyphenolic at varying concentration (0.001-10 μM) for 1 hour. The cells were then rinsed and incubated in the absence of polyphenolics for 24 hours before the addition of ¹²⁵I-labeled Glu-Pmg and fibrinolytic activity using a direct ¹²⁵I-labeled Glu-Pmg activation assay was determined. All red wine derived polyphenolic compounds induced a significant, sustained (24 hours after induction) increase of about 2-3 fold in surface-localized fibrinolytic activity compared to the control cultures. Catechin and epicatechin showed a maximum increase at the lowest concentration (0.001 μM), 91.5±5.0 pmol/well vs. 38.0± pmol/well (p=0.006) and 89.0±4.0 pmol/well vs. 38.0± pmol/well (p=0.008), respectively. Quercetin and resveratrol showed as significant dose-dependent increase in fibrinolytic activity, which peaked at 10 μM (64.0±6.0 vs. 38.0±4.0 (p=0.04) and 55.0±6.0 vs 38.0±4.0 (p=0.02), respectively.

Animal Studies on Rat Aortic Endothelium

For in vivo studies, 18 male Sprague Dawley rats, 250-300 g, were intubated with Popper feeding tubes (20 gauge, 1.5 inches). The control group of three rats were gavaged with PBS in a final volume of 1 ml. Five experimental groups with three rats per groups were gavaged with 1 ml of PBS containing either individual phenolic (catechin, 0.495; epicatechin, 0.224; quercetin, 0.033; and resveratrol, 0.0033 mg/kg body weight), or ethanol 1.25% v/v, for final 0.05% blood level. These doses of phenolics and ethanol are equivalent, on a per kg basis, to the amount consumed in about 2 glasses (25 ml) of red wine by a 70 kg adult. The t-PA and u-PA mRNA expression was analyzed at 6 hours after treatment, when the rats were anesthetized with ketamine/rompin. The thoracic aorta was perfusion-fixed and paraffin embedded to evaluate the effects of ethanol and individual phenolics on the induction of t-PA and u-PA mRNA expression in vivo in the aortic endothelium using in situ hybridization using riboprobes.

To insure that the t-PA and u-PA sense and antisense riboprobes were specific for their respective mRNAs in tissue sections, the sequence-verified riboprobes were hybridized with purified rat liver RNA, rich in t-PA and u-PA mRNA. Both ³²P-labeled t-PA and u-PA antisense riboprobes hybridized with total RNA in a dose-dependent manner, whereas ³²P-labeled t-PA and u-PA sense riboprobes did not hybridize with RNA.

Hybridization of aortic tissue sections from control and treated animals with either t-PA or u-PA biotin-labeled sense riboprobes showed no difference in the intensity of the chromogenic signal, as compared to tissue sections from PBS-treated controls.

Integrity of the aortic endothelium from control and treated animals was verified by examining the autoflourescence of each aortic section. This examination clearly identified individual endothelial cell nuclei, further indicating the intact nature of the endothelium in the processed tissue sections from control and treated animals.

Hybridization or aortic tissue sections from control and treated animals with the t-PA biotin-labeled antisense riboprobe showed more chromogenic signal intensity in aortic tissue sections treated with ethanol and wine phenolics, catechin, epicatechin, quercetin and resveratrol as compared to PBS-treated animals.

In in vivo studies of rat thoracic aorta, eighteen male rats were treated by gavage as indicated above. One control group of three rats was gavaged with 1 ml of saline. Five experimental groups with three rats per group were then gavaged with 1 ml saline containing one of the following: (i) 1.25% (v/v) ethanol, (i) 0.495 mg catechin per kg body weight, (iii) 0.224 mg epicatechin per kg body weight, (iv) 0.033 mg quercetin per kg body weight, or (v) 0.0033 mg resveratrol per kg body weight.

At 3 and 6 hours following gavage, the animals were anesthetized with ketamine/rompin (10 and 1.5 mg/100 g body weight, respectively). The portal vein was cannulated and the vasculature perfused with cold, heparinized saline for 5 minutes to remove blood. The infrarenal inferior was removed to prevent increases in intravascular pressure. The thoracic aorta was perfused-fixed and paraffin embedded to evaluate the short-term effects of alcohol/phenolics on PA1-I mRNA expression using in situ hybridization techniques.

This study demonstrated that 6 hours after in vivo exposure to ethanol or phenolics found in wine, namely, compounds such as catechin, epicatechin, quercetin and resveratrol, there was an altered expression of PAI-1 mRNA in rat aorta ECs. Rat aorta sections hybridized with sense transcript control, in parallel with antisense transcripts for PAI-1, failed to generate a hybridization signal (no chromogenic reaction) in the vascular ECs and SMCs of rat aortas. Integrity of the EC monolayer was demonstrated by digital inversion of the natural autoflourescence of the tissue that demonstrated that the cell nuclei remained intact in the EC after the thoracic aorta perfusion-fixation with 10% formalin and subsequent paraffin embedding. Tissue sections obtained 3 hours after exposure to either moderate alcohol or the polyphenols showed a slight reduction in hybridization signal compared to tissue taken at 6 hours after exposure. Antisense transcript data showed a significant decrease in expression of the PAI-1 mRNA in endothelium of the thoracic aorta in rats that had been exposed to moderate alcohol. Catechin, epicatechin, quercetin and resveratrol all markedly reduced the expression of PAI-1 mRNA in rat aorta endothelial cells.

Because it is not advisable or permitted for many persons to ingest ethanol-containing beverages, it is advantageous to administer separately components contained in wine which have been shown to result in beneficial effects on the cardiovascular system. The administration of polyphenols catechin, epicatechin, resveratrol and quercetin can provide such benefit without exposure to ethanol. The benefit may be obtained by delivery of the polyphenolics separately or in combination. When administered for cardioprotective effect, the daily dosage would be about as follows:

catechin: 5 mg to 1000 mg.,

epicatechin: 2 to 1000 mg.,

quercetin: 2 to 100 mg.,

Resveratrol: 1 to 50 mg.

The more preferred dosage is:

catechin: 5 mg to 100 mg. per day.

epicatechin: 2 to 100 mg. per day.

quercetin: 2 to 10 mg per day.

Resveratrol: 1 to 5 mg per day.

Composition:

A composition comprising, in combination:

10 mg. catechin

5 mg. epicatechin

5 mg. quercetin

2 mg. resveratrol

278 mg. starch

in a capsule.

Composition:

10 mg. catechin

5 mg. epicatechin

5 mg. quercetin

2 mg. resveratrol

in 200 ml. orange juice.

Composition:

catechin: 25 mg.

epicatechin: 10 mg.

Resveratrol: 2 mg.

starch: 63 mg.

Combine and press into a tablet for oral ingestion.

Composition:

Resveratrol: 50 mg.

Quercetin: 50 mg.

Form a suspension in milk or liquid nutritional supplement.

These active agents can be delivered orally individually or in combination by tablet, capsule or in solutions or suspensions with pharmaceutically acceptable carriers. For persons who have difficulty swallowing, it may be wise to give the compositions by dropper or in beverages such as fruit juice, carbonated beverages, etc. While larger dosages may be administered, the dosages suggested are probably sufficient, since they represent, more than the active agent contained in about 2 glasses of wine. The polyphenols may be administered as cyclodextrin inclusion complexes to improve solubility. They may also be added to food supplements and beverages, including nutritionally enriched beverages such as ENSURE™, which are often administered to the elderly or chronically ill persons or are used as meal replacements.

The polyphenols may be given as complexes such as glucuronide, sulfate and methylated conjugates. Complexes formed in the body after ingestion of the polyphenols are highly active.

Quercetin has previously been administered at much higher dosages and is available in dosage of 500 mg. Such high dosage appears to be unnecessary for purposes of cardioprotection, though no ill effects would be expected from ingestion of such amounts.

What we claim is:

1. A composition of matter comprising, as an active agent, at least one of the following:
catechin: 5 mg to 1000 mg.,
epicatechin: 2 to 1000 mg.,
quercetin: 2 to 100 mg., and
Resveratrol: 1 to 50 mg.
in a pharmaceutically acceptable carrier, a beverage, or a nutritional supplement.

2. A composition of claim 1 comprising, in combination,
catechin: 5 mg to 100 mg.,
epicatechin: 2 to 100 mg.,
quercetin: 2 to 10 mg., and
Resveratrol: 1 to 5 mg.
in a pharmaceutically acceptable carrier, a beverage or a nutritional supplement.

3. A method of prophylaxing against the pathologic formation of intravascular thrombi and/or from atherosclerosis, by administration of a composition containing a cardioprotective amount of at least one agent chosen from catechin, epicatechin, quercitin and resveratrol in a pharmaceutically acceptable carrier, a beverage or a nutritional supplement.

4. A method of claim 3 wherein the composition administered contains, in combination,
catechin: 5 mg to 100 mg.,
epicatechin: 2 to 100 mg.,
quercetin: 2 to 10 mg., and
Resveratrol: 1 to 5 mg.

5. A method of providing cardioprotective effects in a patient in need thereof comprising administration of a composition containing a cardioprotective amount of at least one agent chosen from catechin, epicatechin, quercitin and resveratrol in a pharmaceutically acceptable carrier, a beverage or a nutritional supplement.

6. A composition of claim 1 which is a beverage.

7. A composition of claim 1 which is a nutritional supplement.

8. A composition of claim 1 which is a capsule or tablet.

9. A composition of claim 2 which is a beverage.

10. A composition of claim 2 which is a tablet or capsule.

11. A compostion of claim 2 which is a nutritional supplement.
